(19) United States
(12) Patent Application Publication
(10) Pub. No.: US 2024/0139511 A1
(43) Pub. Date: May 2, 2024

(54) INDUCING EYE BLINKING ASSOCIATED WITH VICTIM THROTTLING FOR IMPROVED USER EXPERIENCE

(71) Applicant: QUALCOMM Incorporated, San Diego, CA (US)

(72) Inventors: Karthik RANGARAJU, San Diego, CA (US); Shruti HANUMANTHAIAH, Bangalore (IN); Subbarao LANKA, Bangalore (IN)

(21) Appl. No.: 17/977,283

(22) Filed: Oct. 31, 2022

(51) Int. Cl. A61N 1/36; A61B 5/00; A61B 5/11; A61N 1/04

(52) U.S. Cl. CPC A61N 1/36046; A61B 5/1103; A61B 5/6803; A61N 1/0452

(57) ABSTRACT

A wearable device, including: a display; one or more circuits coupled to the display; a processor configured to: throttle an operation of the one or more circuits; and cause the display to generate an image that induces eye blinking in a user associated with the throttling of the operation of the one or more circuits.

### FIELD

Aspects of the present disclosure relate generally to wearable devices with displays, such as smart glasses, augmented reality viewers or glasses, virtual reality (VR) glasses, intelligent goggles, etc., and in particular, to a wearable device configured to induce eye blinking associated with victim throttling for improved user experience.

### BACKGROUND

Wearable devices, such as smart glasses, augmented reality (AR), virtual reality (VR), or XR (X being a variable for augmented, virtual, and other type) viewers or glasses, fitness measurement and tracking devices, health monitoring devices, medical treatment administering devices, smart watches, and others, are becoming more sophisticated, providing a multitude of applications and functions. As such, wearable devices are designed with increasingly more powerful processors, integrated circuits (IC), system on chips (SOC), and other circuitry, that are capable of implementing multitude of functions at relatively high speeds.

Wearable devices typically have small form factor. As such, they typically include small batteries. Small batteries typically can only outsource a relatively small amount of current, otherwise excessive current may lead to battery brownout (e.g., the battery is not able to produce its specified voltage). Additionally, the small form factor of wearable devices with powerful processors, ICs, SOCs, and other circuitry may generate significant heat, which may be difficult to dissipate because of its relatively small heat sink structure. Thus, controlling current and temperature in a wearable device while not significantly impacting the user experience is of interest.

### SUMMARY

The following presents a simplified summary of one or more implementations in order to provide a basic understanding of such implementations. This summary is not an extensive overview of all contemplated implementations, and is intended to neither identify key or critical elements of all implementations nor delineate the scope of any or all implementations. Its sole purpose is to present some concepts of one or more implementations in a simplified form as a prelude to the more detailed description that is presented later.

An aspect of the disclosure relates to a wearable device. The wearable device includes: a display; one or more circuits coupled to the display; and a processor configured to: trigger a throttling of the one or more circuits or the display; and trigger the display to generate a first image that induces eye blinking in a user associated with the throttling of the one or more circuits or the display.

Another aspect of the disclosure relates to a method of operating a wearable device, including: triggering a throttling of one or more circuits; and inducing eye blinking in a user of the wearable device such that the eye blinking occurs substantially coincidental with a display generating an image including an artifact as a result of the throttling of one or more circuits.

Another aspect of the disclosure relates to an apparatus, including: means for triggering a throttling of one or more circuits; and means for inducing eye blinking in a user such that the eye blinking occurs substantially coincidental with a display generating an image including an artifact as a result of the throttling of one or more circuits.

Another aspect of the disclosure relates to an augmented or virtual reality eyewear, including: a display; a graphics core coupled to the display; a multimedia core coupled to the graphics core; a digital signal processor (DSP) core coupled to the multimedia core; and a processor configured to: trigger a throttling of the one or more of the multimedia core, the graphics core, the DSP core, or the display; and trigger the display to generate a first image that induces eye blinking in a user associated with the throttling of the one or more of the multimedia core, the graphics core, the DSP core, or the display.

To the accomplishment of the foregoing and related ends, the one or more implementations include the features hereinafter fully described and particularly pointed out in the claims. The following description and the annexed drawings set forth in detail certain illustrative aspects of the one or more implementations. These aspects are indicative, however, of but a few of the various ways in which the principles of various implementations may be employed and the description implementations are intended to include all such aspects and their equivalents.

### BRIEF DESCRIPTION OF THE DRAWINGS

FIG. 1 illustrates a perspective view of an example augmented reality (AR) glasses in accordance with an aspect of the disclosure.

FIG. 2A illustrates a block diagram of an example wearable device in accordance with another aspect of the disclosure.

FIGS. 2B-2D illustrate graphs depicting display effects of victim throttling in wearable devices in response to excessive current, temperature, and use case load in accordance with other aspects of the disclosure.

FIG. 3A illustrates a block diagram of another example wearable device in accordance with another aspect of the disclosure.

FIG. 3B illustrates a flow diagram of an example method of inducing eye blinking associated with victim throttling in accordance with another aspect of the disclosure.

FIGS. 3C-3E illustrate graphs depicting effects of inducing eye blinking associated with victim throttling in wearable devices due to excessive current, temperature, and use case load in accordance with other aspects of the disclosure.

FIGS. 4A-4C illustrate graphs depicting time relationships between triggering eye blinking and triggering victim throttling in wearable devices due to excessive current, temperature, and use case budget in accordance with other aspects of the disclosure.

FIG. 4D illustrates a block diagram of another example wearable device in accordance with another aspect of the disclosure.

FIG. 4E illustrates a flow diagram of an example method of inducing eye blinking in a time relationship with victim throttling in response to excess current or temperature in accordance with another aspect of the disclosure.

FIG. 4F illustrates a flow diagram of an example method of inducing eye blinking in a time relationship with victim throttling in response to anticipated excess use case load in accordance with another aspect of the disclosure.

FIG. 5A illustrate timing diagrams related to scheduled eye blinking induction, victim throttling events, and actual eye blinking induction in accordance with another aspect of the disclosure.

FIG. 5B illustrates a flow diagram of an example method of inducing eye blinking based on a scheduled eye blinking induction and victim throttling in accordance with another aspect of the disclosure.

FIG. 6A illustrates a block diagram of another example wearable device in accordance with another aspect of the disclosure.

FIG. 6B illustrates a flow diagram of an example method of conditional eye blinking induction associated with victim throttling in accordance with another aspect of the disclosure.

FIG. 7A illustrates a block diagram of another example wearable device in accordance with another aspect of the disclosure.

FIG. 7B illustrates a flow diagram of an example method of initiating or increasing foveate rendering in response to frequent eye blinking inductions due to victim throttling in accordance with another aspect of the disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. However, it will be apparent to those skilled in the art that these concepts may be practiced without these specific details. In some instances, well-known structures and components are shown in block diagram form in order to avoid obscuring such concepts.

As discussed, wearable devices typically include a relatively small battery due to their usually small form factor. For example, batteries used in augmented reality (AR) viewers or glasses, virtual reality (VR) viewers or glasses, or XR (e.g., where X is a variable for augmented, virtual, and other type of) viewers or glasses may typically have a current-time rating of around 200 milli Ampere hour (MAH). In certain use cases, the current demand from such batteries due to the various circuits, including processors, cores, peripherals, and other circuitry in wearable devices may cause the maximum current rating of such batteries to be exceeded; which may lead to battery brownout (e.g., the battery not being able to produce its rated voltage).

Further, these wearable devices may employ a set of power or supply voltage rails configured to provide power/current to various circuits, including processors, cores, peripherals and other circuitry. In some cases, these wearable device includes a power management integrated circuit (PMIC) including a set of voltage regulators coupled to the set of power rails, respectively. A battery provides an input voltage to the PMIC from which it generates a set of supply voltages for the set of power rails, respectively. The set of power rails of the PMIC may also have a set of maximum current ratings that should not be exceeded, at least for an extended period, or otherwise damage may occur in the PMIC. Apart from the power rails, the set of circuits, such as processors, cores, peripherals, and other circuitry in a wearable device may also have their respective maximum current ratings.

An additional consideration in wearable devices is heat generated by the circuits therein. As these devices are worn by users, the heat generated by such wearable devices should be considered as they are often in contact with users' skin, and the heat generated may be uncomfortable, or in some cases, may cause harm to users. Further, heat generated by such wearable devices may also have adverse impact on the internal components of wearable devices. Accordingly, battery current, PMIC regulator power rail currents, currents consumed by circuits, such as processors, cores, peripherals, and other circuitry in wearable devices, and temperature generated by wearable devices, should be monitored and action taken when such currents and/or temperature exceed prescribed thresholds.

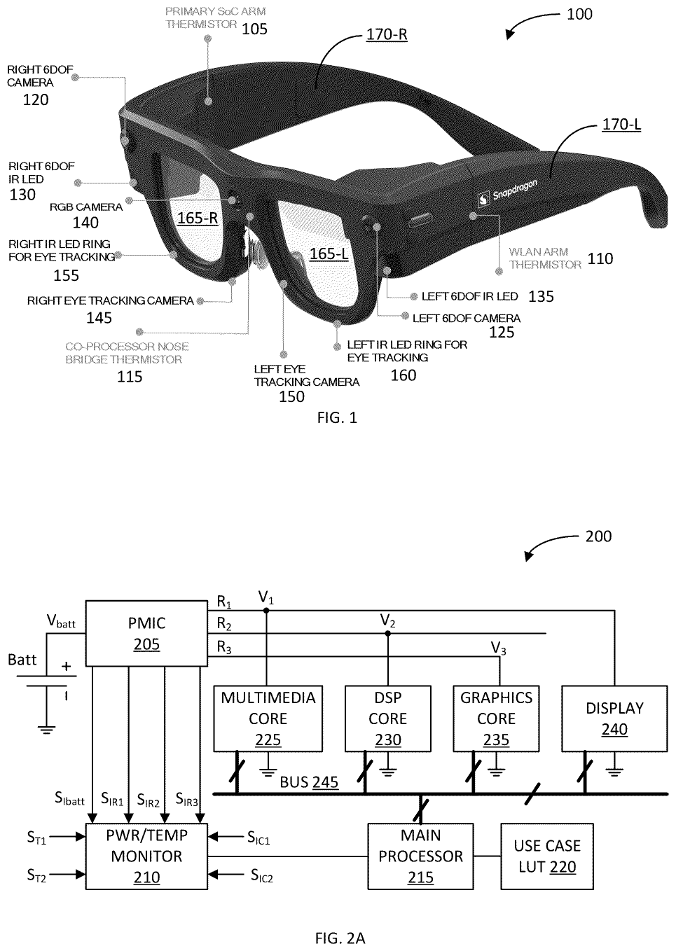

FIG. 1 illustrates a perspective view of an example augmented reality (AR) glasses 100 in accordance with an aspect of the disclosure. As previously discussed, the AR glasses 100 is an example of a wearable device. Further, as previously mentioned, it shall be understood that a wearable device described herein may take on many different forms, such as XR viewers, glasses, goggles, fitness measurement and tracking devices, health monitoring devices, medical treatment devices, smart watches, and others.

In this example, the AR glasses 100 includes right and left lens displays 165-R and 165-L, and right and left audio circuits/speakers 170-R and 170-L. The AR glasses 100 further includes a set of skin temperature sensors 105, 110, and 115. The skin temperature sensor 105 may be positioned on the right temple of the AR glasses 100. The skin temperature sensor 110 may be positioned on the left temple of the AR glasses 100. The skin temperature sensor 115 may be positioned on the nose bridge of the AR glasses 100.

The AR glasses 100 further includes right and left 6 degree of freedom (DOF) cameras 120 and 125 pointing generally forward, and situated on the exterior right and left rims near the right and left hinges of the AR glasses 100, respectively. The AR glasses 100 also includes right and left infrared (IR) LEDs 130 and 135 also pointing generally forward, and situated on the exterior right and left rims below the right and left 6DOF cameras 120 and 125, respectively. Further, the AR glasses 100 includes a video (red, green, blue (RGB)) camera 140 pointing generally forward, and situated on the nose bridge of the AR glasses 100.

For eye tracking, the AR glasses 100 includes right and left eye tracking cameras 145 and 150 pointing in the directions of the right and left eyes of a user when the AR glasses are worn, and situated on the bridge interior sides of the right and left rims, respectively. Further, the AR glasses 100 includes right and left infrared (IR) LED rings (e.g., series-connected LEDs) 155 and 160 for illuminating the right and left eye regions of a user when the AR glasses are worn, and situated along the interior surfaces of the right and left rims, respectively. It shall be understood that the aforementioned components, placements, and orientations are merely examples, and such configuration of an AR glasses may take on many different forms.

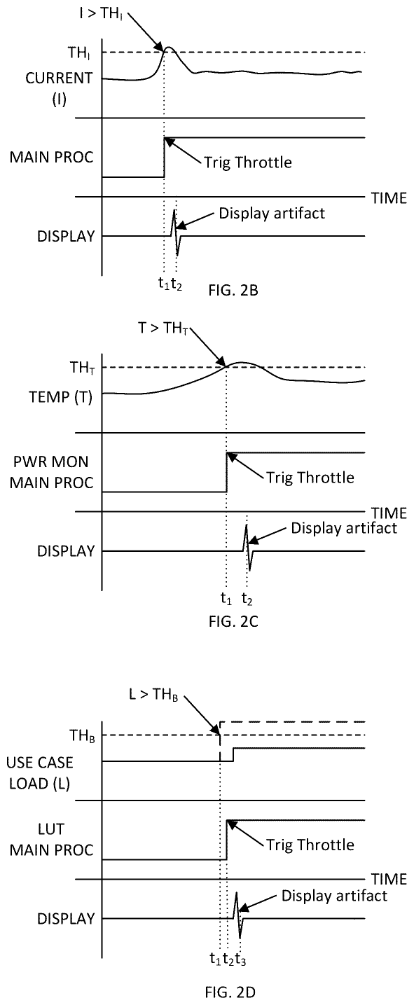

FIG. 2A illustrates a block diagram of an example wearable device 200 in accordance with another aspect of the disclosure. The wearable device 200 may be an example implementation of the AR glasses 100 previously discussed. The wearable device 200 includes a battery (Batt), a power management integrated circuit (PMIC) 205, a power/temperature monitor 210, and a main processor 215 (e.g., a central processing unit (CPU)) including an associated use case budget look-up table (LUT) 220. Additionally, the wearable device 200 may include one or more circuits, such as a set of cores (e.g., a multimedia core 225 (e.g., 6DOF cameras, video cameras, displays, etc.), a digital signal processing (DSP) (sometimes referred to as a compute or data processing unit (DPU)) core 230, and a graphics core 235). Further, the wearable device 200 may include one or more peripherals, such as a display 240 (which may be part of the multimedia core, but shown separately for description purposes).

It shall be understood that the wearable device 200 is merely an example, and its component configuration may vary significantly. For example, although not shown, the one or more circuits of the wearable device 200 may further include an applications processor, an engine for visual analysis (EVA), a modem/transceiver for communication via a wireless local area network (e.g., WiFi), wireless wide area network (WWAN) (e.g., Long Term Evolution (LTE), fifth generation (5G) New Radio (NR), etc.), or other.

The battery is configured to generate and provide a battery voltage V_{batt} to the PMIC 205. The PMIC 205 is configured to generate a set of supply voltages V₁, V₂, and V₃ for a set of power rails R₁, R₂, and R₃, respectively. Although three (3) supply voltages and power rails are shown for description purposes, it shall be understood that the wearable device 200 may include any number of supply voltages and power rails. In this example, the multimedia core 225 and the display 240 may be coupled between the power rail R₁ and a lower voltage rail (e.g., ground). The DSP core 230 may be coupled between the power rail R₂ and the lower voltage rail. The graphics core 235 may be coupled between the power rail R₃ and the lower voltage rail. Although not shown, it shall be understood that the power/temperature monitor 210, main processor 215, and use case budget LUT 220 may also be coupled to one or more power rails.

As previously discussed, the battery current provided to the PMIC 205 by the battery may be monitored so that it does not exceed a maximum current rating or threshold. Similarly, the rail currents provided by the PMIC 205 to the multimedia core 225/display 240, DSP core 230, and graphics core 235 by generating the supply voltages V₁ to V₃ on power rails R₁ to R₃ may be monitored so that they do not exceed their respective maximum current ratings or thresholds. In a like manner, the core currents consumed by the multimedia core 225, the DSP core 230, and graphics core 235 (as these currents may be different from the corresponding rail currents as there may be other circuits (not shown) coupled to the power rails R₁ to R₃) may be monitored so that they do not exceed their respective maximum current ratings or thresholds. Finally, in this example, the temperature near the skin of a user as well as circuit (e.g., junction) temperature at various hot spots within the cores 225, 230, and 235 may be monitored so that they do not exceed their respective maximum temperature ratings or thresholds.

For current monitoring purpose, the power/temperature monitor 210 may receive a signal S_{Ibatt} from the PMIC 205 (or other current sensor) indicative of the battery current. The power/temperature monitor 210 may also receive a set of signals S_{IR1}, S_{IR2}, and S_{IR3} from the PMIC 205 (or other current sensors) indicative of the rail currents supplied by the set of power rails R₁, R₂, and R₃, respectively. The power/temperature monitor 210 may further receive a set of signals S_{IC1} and S_{IC2} (two in this example, but could be different) from the corresponding cores 225, 230, and/or 235 (or other current sensors) indicative of the core load currents, respectively. For temperature monitoring purpose, the power/temperature monitor 210 may further receive a set of signals S_{T1} and S_{T2} (two in this example, but could be different) from skin temperature sensor and circuit temperature sensor, respectively.

The power/temperature monitor 210 may compare the signals S_{Ibatt}, S_{IR1}, S_{IR2}, S_{IR3}, S_{IC1}, S_{IC2}, S_{T1}, and S_{T2} to corresponding maximum current/temperature thresholds, and issue a throttling interrupt provided to the main processor 215 when any of these signals indicate current/temperature exceeding the corresponding threshold(s). In response to the throttling interrupt, the main processor 215 may send signals to the multimedia core 225, DSP core 230, the graphics core 235, and the display 240 via a data bus 245 to throttle their respective operations so as to lower the current/temperature such that they are reduced below the corresponding maximum current/temperature thresholds. The one or more circuits, such as the core(s), peripheral(s), processor(s), and/or circuitry, subjected to throttling may be referred to herein as the “victim(s).” Although a single data bus 245 is shown for illustrative purposes, it shall be understood that the wearable device 200 may include a set of data buses for coupling the various circuits together.

As some examples, the main processor 215 may trigger the throttling of a victim by causing its supply voltage to be reduced (e.g., reducing supply voltage V₁ provided to the multimedia core 225), and/or causing a frequency of a clock signal driving the operations of a core to be reduced (e.g., reducing a frequency f₁ of a clock signal provided to the multimedia core 225), and/or reducing operation parameters of peripherals (e.g., reducing a resolution and/or brightness of the display 240). It shall be understood that the aforementioned victim throttling are merely some examples, and the nature, extent, and number of victims involved in the throttling may vary significantly.

The aforementioned victim throttling may be referred to as “reactive” victim throttling, where the sensed current and/or temperature may exceed the maximum current and/or temperature thresholds, and the main processor 215 reactively or responsively triggers the throttling of the victims to reduce the sensed current and/or temperature below the maximum current and/or temperature thresholds, respectively.

Another approach to victim throttling may be proactive. For various use cases, the main processor 215 may keep track of the use case load of the various circuits (e.g., cores 225, 230, and 235), and when a request for increased functionality/operations of the cores 225, 230, and 235 is provided to the main processor 215, the main processor 215 accesses the use case budget LUT 220 to determine whether the increased functionality/operations of one or more of the cores 225, 230, and 235 would result in the use case load (power/current) exceeding the use case budget. If it does not, in accordance with the use case budget LUT 220, the main processor 215 honors the request and configures the one or more of the cores 225, 230, and 235 to operate in accordance with the increased functionality/operations. However, if the increased functionality/operations of the one or more of the cores 225, 230, and 235 would result in the use case load exceeding the use case budget in accordance with the use case budget LUT 220, the main processor 215 triggers the throttling of one or more victims so that the request may be honored without exceeding the use case budget.

FIG. 2B illustrate a graph depicting display effects of victim throttling in the wearable device 200 in response to a sensed current exceeding a maximum current rating or threshold in accordance with another of the disclosure. The x- or horizontal-axis of the graph represents time. The y- or vertical axis of the graph, from top to bottom, represents: a sensed current “I” (e.g., the battery current, a rail current, a core current, etc.) including a horizontal dashed line indicating a corresponding maximum current rating or threshold TH_{I}; the signal generated by the main processor 215 to trigger victim throttling (e.g., a logic low state indicates no victim throttling, a high logic state indicates victim throttling); and the effects on an image (e.g., motion or still) rendered by the display 240.

For example, prior to time t₁, the sensed current I is below the current threshold TH_{I}, and therefore, the main processor 215 generates a deasserted victim throttling trigger signal (e.g., at a logic low state), and the display 240 renders the image without any artifacts (e.g., image distortion, glitches, jitter, image tearing, etc.), as represented by a straight horizontal line. At time t₁, the sensed current I crosses (exceeds) the current threshold TH_{I}, and the main processor 215 asserts the victim throttling trigger signal (e.g., at a logic high state), and after a slight delay at time t₂, when the victim throttling occurs, the display 240 produces an image artifact (e.g., image distortion, glitch, jitter, image tearing, etc.), as represented by a fluctuation. In response to the victim throttling after time t₂, the sensed current I decreases below the current threshold TH_{I} for safe operations thereafter, where the display 240 again renders the image without any artifacts.

FIG. 2C illustrate a graph depicting display effects of victim throttling in the wearable device 200 in response to a sensed temperature exceeding a maximum temperature rating or threshold in accordance with another of the disclosure. The x- or horizontal-axis of the graph represents time. The y- or vertical axis of the graph, from top to bottom, represents: a sensed temperature “T” (e.g., a user skin temperature, a circuit (e.g., junction) temperature, etc.) including a horizontal dashed line indicating a corresponding maximum temperature rating or threshold TH_{T}; the signal generated by the main processor 215 to trigger victim throttling (e.g., a logic low state indicates no victim throttling, a high logic state indicates victim throttling); and the effects on an image (e.g., motion or still) rendered by the display 240.

For example, prior to time t₁, the sensed temperature T is below the temperature threshold TH_{T}, and therefore, the main processor 215 generates a deasserted victim throttling trigger signal (e.g., at a logic low state), and the display 240 renders the image without any artifacts (e.g., image distortion, glitch, jitter, image tearing, etc.), as represented by a straight horizontal line. At time t₁, the sensed temperature T crosses (exceeds) the temperature threshold TH_{T}, and the main processor 215 asserts the victim throttling trigger signal (e.g., at a logic high state), and after a slight delay at time t₂, when the victim throttling occurs, the display 240 produces an image artifact (e.g., image distortion, glitch, jitter, image tearing, etc.), as represented by a fluctuation. In response to the victim throttling after time t₂, the sensed temperature T decreases below the temperature threshold TH_{T} for safe operations thereafter, where the display 240 again renders the image without any artifacts.

FIG. 2D illustrate a graph depicting deplay effects of victim throttling in the wearable device 200 in response to a use case load exceeding a use case budget in accordance with another of the disclosure. The x- or horizontal-axis of the graph represents time. The y- or vertical axis of the graph, from top to bottom, represents: use case load (L) (e.g., the use case load demand from the one or more cores, peripherals, processors and/or other circuitry) including a horizontal small dashed line indicating the use case budget TH_{B}; the signal generated by the main processor 215 to trigger victim throttling (e.g., a logic low state indicates no victim throttling, a high logic state indicates victim throttling); and the effects on an image (e.g., motion or still) rendered by the display 240.

For example, prior to time t₁, the use case load (L), according to the use case budget LUT 220, is below the use case budget TH_{B}, and therefore, the main processor 215 generates a deasserted victim throttling trigger signal (e.g., at a logic low state), and the display 240 renders the image without any artifacts (e.g., image distortion, glitch, jitter, image tearing, etc.), as represented by a straight horizontal line. At time t₁, a request is provided to the main processor 215 for increased functionality/operations of the one or more of the cores, peripherals, processors and/or other circuitry. In response to the request, the main processor 215 accesses the use case budget LUT 220 to determine whether the request can be honored without victim throttling. In this example, the main processor 215 may not honor the request without victim throttling. As shown by the large dash line, if the main processor 215 does not initiate victim throttling when honoring the request, the use case load (L) would exceed the use case budget TH_{B}.

Accordingly, at time t₂, the main processor 215 triggers victim throttling by asserting the victim throttling trigger signal (e.g., at a logic high state), and after a slight delay at time t₃, when the victim throttling occurs, the display 240 produces an image artifact (e.g., image distortion, glitch, jitter, image tearing, etc.), as represented by a fluctuation. In response to the victim throttling after time t₃, the use case load (L) continues or is maintained below the use case budget TH_{B} for safe operations thereafter, where the display 240 again renders the image without any artifacts.

The aforementioned image artifacts generally degrade the user experience. That is, victim throttling may occur often when the wearable device 200 is performing large amounts of data processing at rates needed to provide acceptable user experience. As a result of such victim throttling, a user may frequently experience display or image artifacts, and may be annoying and distracting; thereby, degrading the user experience with the wearable device. The following describes wearable devices and techniques for dealing with display or image artifacts due to victim throttling to improve user experience with wearable devices.

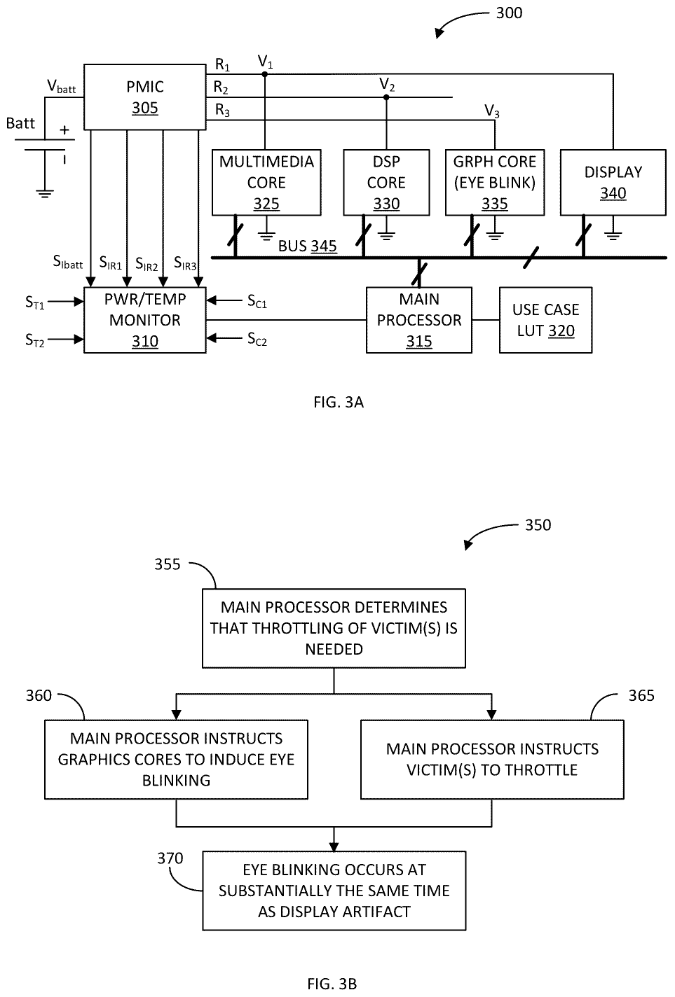

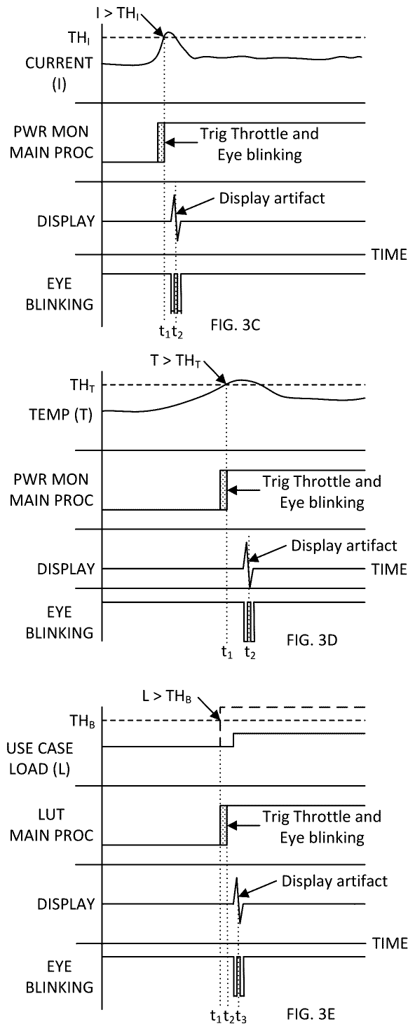

FIG. 3A illustrates a block diagram of another example wearable device 300 in accordance with another aspect of the disclosure. As discussed in more detail herein, the wearable device 300 induces eye blinking substantially coincidental with victim throttling so that the eye blinking and the display or image artifacts occur at substantially the same time. If a user is blinking his or her eyes during the display of image artifacts, then the user will not be able to observe the image artifacts; and thus, from a user's perspective, it is as if the image artifacts did not occur. This would significantly improve the user experience because effectively the eye blinking masks the image artifacts.

In particular, the wearable device 300 is similar to wearable device 200 previously discussed in detail, with similar elements identified by the same reference numbers with the exception that the most significant digit is a “3” in wearable device 300 compared to a “2” in wearable device 200. More specifically, the wearable device 300 includes a battery (Batt), a PMIC 305, a power/temperature monitor 310, a main processor 315, a use case budget LUT 320, one or more circuits, such as a set of cores (e.g., a multimedia core 325, a DSP core 330, and a graphic core 335), and a set of one or more peripherals, such as a display 340. The wearable device 300 further includes a data bus 345 communicatively coupling together the multimedia core 325, DSP core 330, and graphics core 335, the display 340, and main processor 315. The operation of these elements with regard to victim throttling has been previously discussed in detail with reference to wearable device 200.

FIG. 3B illustrates a flow diagram of an example method 350 of inducing eye blinking associated with (e.g., in response to or in anticipation of) victim throttling in accordance with another aspect of the disclosure. The method 350 may be implemented by the main processor 315 of wearable device 300.

According to the method 350, the main processor 315 determines that throttling of one or more victims is needed (block 355). As previously discussed, the main processor 315 may determine that throttling of one or more victims is needed based on an interrupt received from the power/temperature monitor 310 in response to the latter determining that one or more of the signals S_{Ibatt}, S_{IR1}, S_{IR2}, S_{IR3}, S_{C1}, and S_{C2} indicate corresponding one or more currents exceeding the corresponding one or more maximum current ratings or thresholds and/or the signals S_{T1} and S_{T2} indicate corresponding one or more temperatures exceeding the corresponding one or more maximum temperature ratings or thresholds.

The method 350 further includes the main processor 315 instructing the graphics core 335 (e.g., via the data bus 345) to provide an image signal to the display 340 (e.g., via the data bus 345) to induce eye blinking in a user associated with the determination that one or more victims are to be throttled in block 355 (block 360). For example, in response to the eye blinking inducing image signal, the display 340 may produce an image where its peripheral region is blurred momentarily, which induces eye blinking in the user. It shall be understood that eye blinking induction image generated by the display 340 may take on various different forms.

Additionally, the method 350 includes the main processor 315 instructing the one or more victims (e.g., via the data bus 345) to throttle (block 365). As previously discussed, the one or more victims may be throttled by providing a lower supply voltage to the one or more victims, and/or providing one or more clock signals driving the operations of the one or more victims with reduced frequency, and/or reducing the functionality/operations performed by the one or more victims (e.g., reducing the resolution and/or brightness of the display 340).

As a result of the eye blinking induction in block 360 and the victim throttling in block 365, the method 350 includes the user blinking his/her eyes at substantially the same time as the display or image artifact is rendered (block 370). The eye blinking by the user at substantially the same time as the display or image artifacts results in the user not experiencing the display or image artifact; thereby, improving the user experience with the wearable device 300. In other words, from the user's perspective, the eye blinking masks the display or image artifact.

FIG. 3C illustrate graphs depicting effects of inducing eye blinking associated with victim throttling in wearable devices due to excessive current in accordance with another aspect of the disclosure. The x- or horizontal-axis of the graph represents time. The y- or vertical axis of the graph, from top to bottom, represents: the sensed current “I” (e.g., the battery current, a rail current, a core current, etc.) including a horizontal dashed line indicating a corresponding maximum current rating or threshold the signal(s) generated by the main processor 315 to trigger victim throttling and eye blinking (e.g., a logic low state indicates no victim throttling and eye blinking, a high logic state indicates victim throttling and eye blinking); the effects on an image (e.g., motion or still) rendered by the display 340; and the eye blinking induced in the user.

For example, prior to time t₁, the sensed current I is below the current threshold TH_{I}, and therefore, the main processor 315 generates deasserted victim throttling and eye blinking induction signal(s) (e.g., at a logic low state), and the display 340 renders the image without any artifacts (e.g., image distortion, glitch, jitter, image tearing, etc.), as represented by a straight horizontal line. At time t₁, the sensed current I crosses (exceeds) the current threshold TH_{I}, and the main processor 315 asserts the victim throttling and eye blinking induction signal(s) (e.g., at a logic high state). Note that there is an interval represented by a shaded area before time t₁ to indicate that the triggering of victim throttling and eye blinking need not occur at the same time. As discussed further herein, the triggering of eye blinking may occur before (in anticipation of) the triggering of victim throttling so that eye blinking and the display or image artifact as a result of victim throttling occur substantially at the same time.

Then, after a slight delay from time t₁, at time t₂ the display 340 produces an image artifact (e.g., image distortion, glitch, jitter, image tearing, etc.) as a result of the victim throttling, as represented by a fluctuation. Also, at substantially time t₂, the user blinks his/her eyes as a result of the eye blinking induction, as indicated by the square shape fluctuations. The eye blinking by the user at substantially the same time as the display or image artifacts results in the user not experiencing the display or image artifact; thereby, improving the user experience with the wearable device 300. In other words, from the user's perspective, the eye blinking masks the display or image artifact. Then, in response to the victim throttling after time t₂, the sensed current I decreases below the current threshold TH_{I} for safe operations thereafter, where the display 340 again renders the image without any artifacts.

FIG. 3D illustrate graphs depicting effects of eye blinking associated with victim throttling in wearable devices due to excessive temperature in accordance with other aspects of the disclosure. The x- or horizontal-axis of the graph represents time. The y- or vertical axis of the graph, from top to bottom, represents: a sensed temperature “T” (e.g., a user skin temperature, a circuit (e.g., junction) temperature, etc.) including a horizontal dashed line indicating a corresponding maximum temperature rating or threshold TH_{T}; the signal(s) generated by the main processor 315 to trigger victim throttling and eye blinking (e.g., a logic low state indicates no victim throttling and eye blinking, a high logic state indicates victim throttling and eye blinking); the effects on an image (e.g., motion or still) rendered by the display 340; and the eye blinking induced in the user.

For example, prior to time t₁, the sensed temperature T is below the temperature threshold TH_{T}, and therefore, the main processor 315 generates deasserted victim throttling and eye blinking induction signal(s) (e.g., at a logic low state), and the display 340 renders the image without any artifacts (e.g., image distortion, glitch, jitter, image tearing, etc.), as represented by a straight horizontal line. At time t₁, the sensed temperature T exceeds the temperature threshold TH_{T}, and the main processor 315 asserts the victim throttling and eye blinking induction signal(s) (e.g., at a logic high state). Note that there is an interval represented by a shaded area before time t₁ to indicate that the triggering of victim throttling and eye blinking need not occur at the same time. As discussed further herein, the triggering of eye blinking may occur before (in anticipation of) the triggering of victim throttling so that eye blinking and the display or image artifact as a result of victim throttling occur substantially at the same time.

Then, after a slight delay from time t₁, at time t₂ the display 340 produces an image artifact (e.g., image distortion, glitch, jitter, image tearing, etc.) as a result of the victim throttling, as represented by a fluctuation. Also, at substantially time t₂, the user blinks his/her eyes as a result of the eye blinking induction, as indicated by the square shape fluctuations. The eye blinking by the user at substantially the same time as the display or image artifacts results in the user not experiencing the display or image artifact; thereby, improving the user experience with the wearable device 300. In other words, from the user's perspective, the eye blinking masks the display or image artifact. Then, in response to the victim throttling after time t₂, the sensed temperature T decreases below the temperature threshold TH_{T} for safe operations thereafter, where the display 340 again renders the image without any artifacts.

FIG. 3E illustrate graphs depicting effects of eye blinking associated with victim throttling in wearable devices due to excessive temperature in accordance with other aspects of the disclosure. The x- or horizontal-axis of the graph represents time. The y- or vertical axis of the graph, from top to bottom, represents: use case load (L) (e.g., the use case load demand from the one or more cores, peripherals, processors and/or other circuitry) including a horizontal small dashed line indicating a use case load budget threshold TH_{B}; the signal(s) generated by the main processor 315 to trigger victim throttling and eye blinking (e.g., a logic low state indicates no victim throttling and eye blinking, a high logic state indicates victim throttling and eye blinking); the effects on an image (e.g., motion or still) rendered by the display 340; and the eye blinking induced in the user.

For example, prior to time t₁, the current use case load (L), according to the use case budget LUT 320, is below the use case budget threshold TH_{B}, and therefore, the main processor 315 generates deasserted victim throttling and eye blinking induction signal(s) (e.g., at a logic low state), and the display 340 renders the image without any artifacts (e.g., image distortion, glitch, jitter, image tearing, etc.), as represented by a straight horizontal line. At time t₁, a request is provided to the main processor 315 for increased functionality/operation of the one or more of the cores, peripherals, processors and/or other circuitry. In response to the request, the main processor 315 accesses the use case budget LUT 320 to determine whether the request can be honored without victim throttling. In this example, the main processor 315 cannot be honored without victim throttling. As shown by the large dash line, if the main processor 315 does not initiate victim throttling when honoring the request, the use case load (L) would exceed the use case budget threshold TH_{B}.

Accordingly, at time t₂, the main processor 315 asserts the victim throttling and eye blinking induction signal(s) (e.g., at a logic high state). Note that there is an interval represented by a shaded area before time t₂ to indicate that the triggering of victim throttling and eye blinking need not occur at the same time. As discussed further herein, the triggering of eye blinking may occur before (in anticipation of) the triggering of victim throttling so that eye blinking and the display or image artifact as a result of victim throttling occur substantially at the same time.

Then, after a slight delay from time t₂, at time t₃ the display 340 produces an image artifact (e.g., image distortion, glitch, jitter, image tearing, etc.) as a result of the victim throttling, as represented by a fluctuation. Also, at substantially time t₃, the user blinks his/her eyes as a result of the eye blinking induction, as indicated by the square shape fluctuations. The eye blinking by the user at substantially the same time as the display or image artifacts results in the user not experiencing the display or image artifact; thereby, improving the user experience with the wearable device 300. In other words, from the user's perspective, the eye blinking masks the display or image artifact. Then, in response to the victim throttling after time t₂, the use case load (L) continues or is maintained below the use case budget threshold TH_{B} for safe operations thereafter, where the display 340 again renders the image without any artifacts.

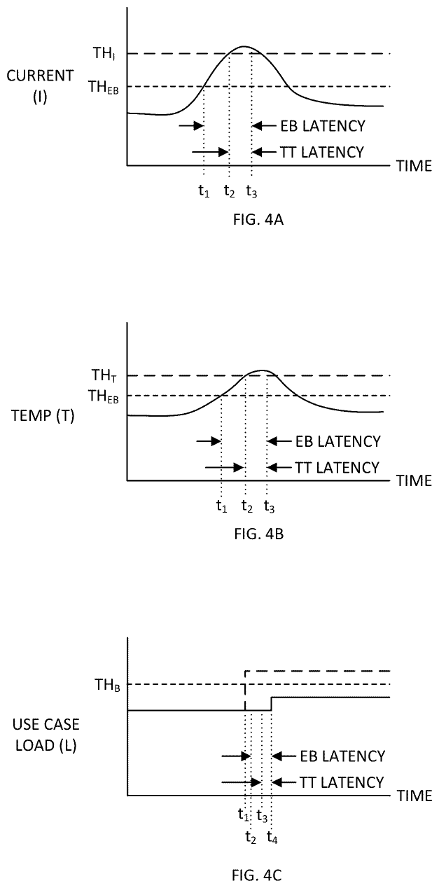

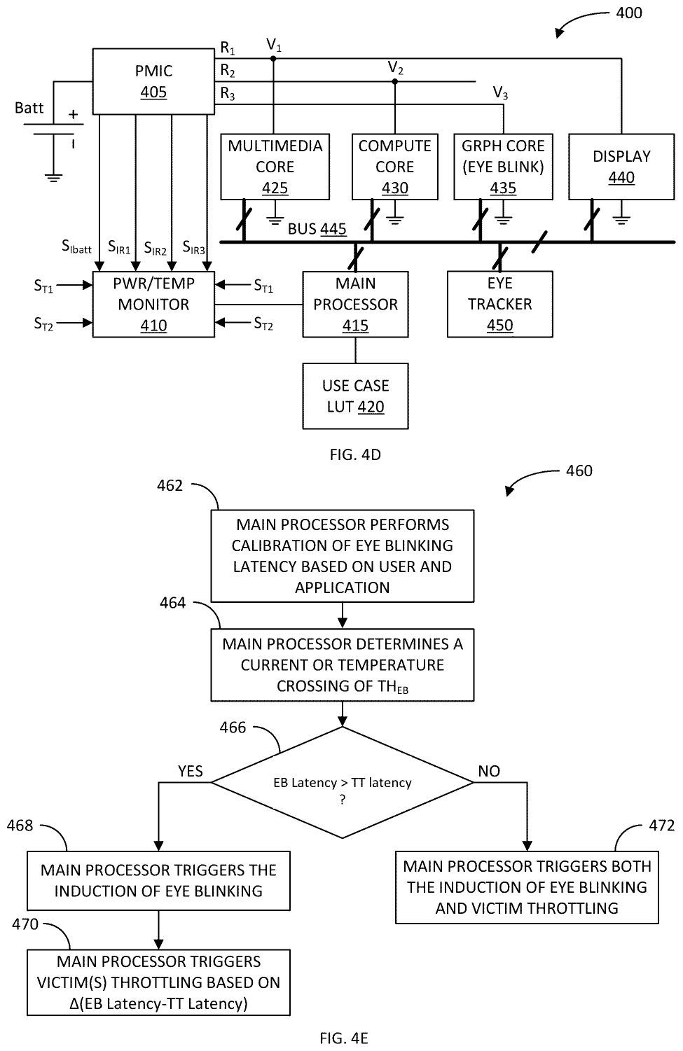

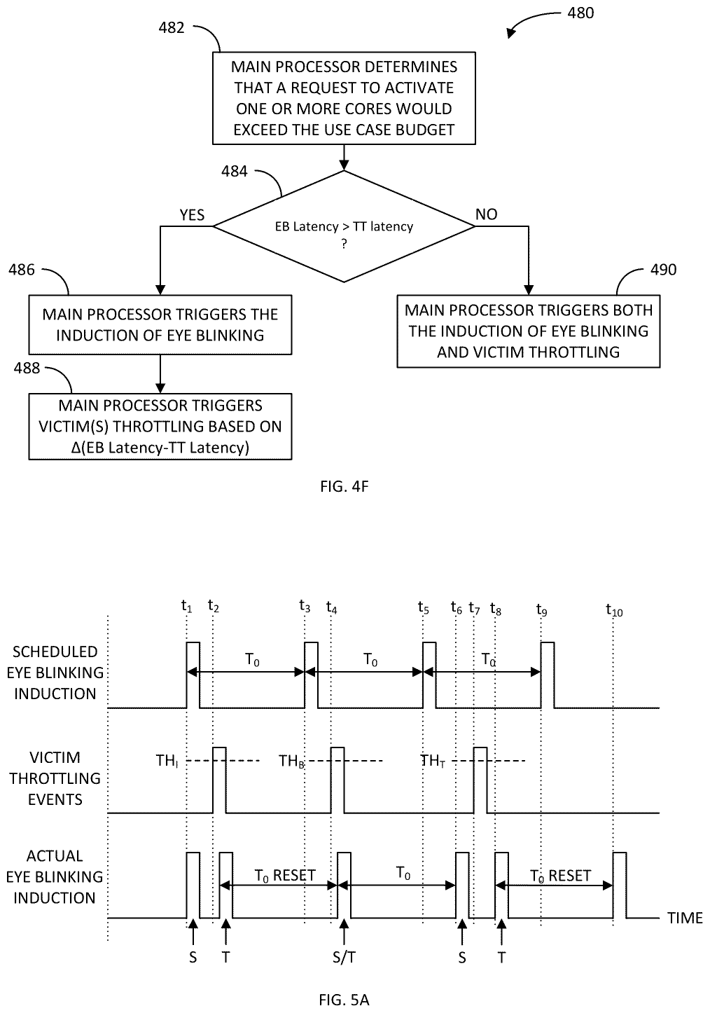

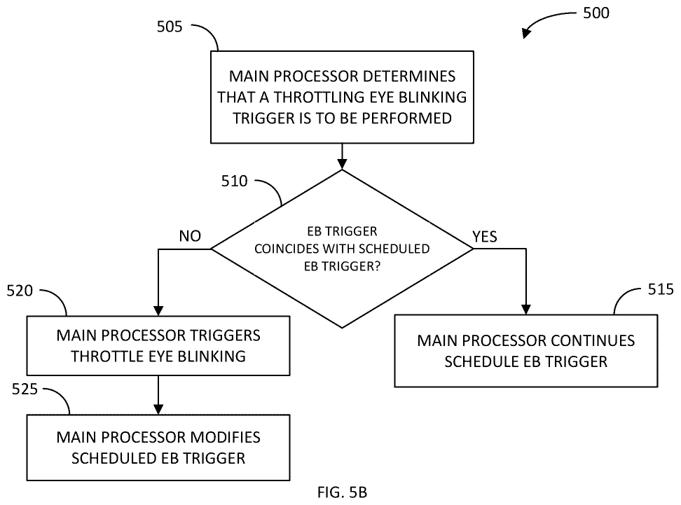

FIG. 4A illustrates a graph depicting a time relationship between triggering eye blinking and triggering victim throttling in a wearable device due to excessive current in accordance with other aspects of the disclosure. The x- or horizontal-axis of the graph represents time. The y- or vertical axis of the graph represents the sensed current “I” (e.g., the battery current, a rail current, a core current, etc.) including a horizontal short-dashed line indicating a threshold current TH_{EB}, where eye blinking is to be induced or triggered, and a horizontal long-dashed line corresponding to a maximum current rating or threshold TH_{I}.

As previously discussed, it may be desired that the eye blinking in the user occurs substantially at the same time as the display or image artifact so that the user does not perceive the display or image artifact. The latency between the triggering of the eye blinking to the time the user blinks his/her eyes may be indicated as EB latency. The latency between the triggering of the victim throttling and the actual victim throttling when the display or image artifact occurs may be indicated as TT latency. If the EB latency is different than the TT latency, and both the eye blinking and victim throttling are triggered at the same time, then the eye blinking by the user may not occur at substantially the same time as the display or image artifact. In such case, the user would perceive the display or image artifact, and the user experience with the wearable device may be impacted.

In the example depicted in the graph of FIG. 4A, the EB latency is greater than the TT latency. Thus, in order for the eye blinking by the user to occur at substantially the same time as the display or image artifact, the triggering of the eye blinking should occur before (e.g., in anticipation of) the triggering of the victim throttling. To effectuate the triggering of the eye blinking before the triggering of the victim throttling, the eye blinking current threshold TH_{EB} is set lower than maximum current rating or threshold TH_{I}. The difference between the eye blinking threshold current TH_{EB} and the maximum current rating or threshold TH_{I} is related to the difference in EB and TT latencies.

Thus, at time t₁, the sensed current I crosses (exceeds) the eye blinking threshold TH_{EB}. In response, the main processor 315 instructs the graphics core 335 (e.g., via the data bus 345) to generate and provide an image signal to the display 340 (e.g., via the data bus 345) that renders an image that induces eye blinking by the user. Then, at time t₂, the sensed current I crosses (exceeds) the maximum current rating or threshold TH_{I}. In response, the main processor 315 instructs the one or more victims to throttle. Then, at time t₃, the user blinks his/her eyes at substantially the same time as the display or image artifact is rendered due to victim throttling. Thus, the user may not perceive the display or image artifact; and accordingly, the user experience with the wearable device 300 is improved.

FIG. 4B illustrates a graph depicting a time relationship between triggering eye blinking and triggering victim throttling in a wearable device due to excessive temperature in accordance with another aspect of the disclosure. The x- or horizontal-axis of the graph represents time. The y- or vertical axis of the graph represents the sensed temperature “T” (e.g., a user skin temperature, a circuit (e.g., junction) temperature, etc.) including a horizontal short-dashed line indicating a threshold current TH_{EB}, where eye blinking is to be induced or triggered, and a horizontal long-dashed line corresponding to maximum temperature rating or threshold TH_{T}.

As previously discussed, it may be desired that the eye blinking in the user occurs substantially at the same time as the display or image artifact so that the user does not perceive the display or image artifact. The latency between the triggering of the eye blinking to the time the user blinks his/her eyes may be indicated as EB latency. The latency between the triggering of the victim throttling and the actual victim throttling when the display or image artifact occurs may be indicated as TT latency. If the EB latency is different than the TT latency, and both the eye blinking and victim throttling are triggered at the same time, then the eye blinking by the user may not occur at substantially the same time as the display or image artifact. In such case, the user would perceive the display or image artifact, and the user experience with the wearable device may be impacted.

In the example depicted in the graph of FIG. 4B, the EB latency is greater than the TT latency. Thus, in order for the eye blinking by the user to occur at substantially the same time as the display or image artifact, the triggering of the eye blinking should occur before (e.g., in anticipation of) the triggering of the victim throttling. To effectuate the triggering of the eye blinking before the triggering of the victim throttling, the eye blinking temperature threshold TH_{EB} is set lower than maximum temperature rating or threshold TH_{T}. The difference between the eye blinking temperature threshold TH_{EB} and the maximum temperature rating or threshold TH_{T} is related to the difference in EB and TT latencies.

Thus, at time t₁, the sensed temperature T crosses (exceeds) the eye blinking threshold TH_{EB}. In response, the main processor 315 instructs the graphics core 335 (e.g., via the data bus 345) to generate and provide an image signal to the display 340 (e.g., via the data bus 345) that renders an image that induces eye blinking by the user. Then, at time t₂, the sensed temperature T crosses (exceeds) the maximum temperature rating or threshold TH_{T}. In response, the main processor 315 instructs one or more victims to throttle. Then, at time t₃, the user blinks his/her eyes at substantially the same time as the display or image artifact is rendered due to victim throttling. Thus, the user may not perceive the display or image artifact; and accordingly, the user experience with the wearable device 300 is improved.

FIG. 4C illustrates a graph depicting a time relationship between triggering eye blinking and triggering victim throttling in a wearable device due to excessive use case load in accordance with another aspect of the disclosure. The x- or horizontal-axis of the graph represents time. The y- or vertical axis of the graph represents the use case load (L) (e.g., the use case load demand of the one or more cores, peripherals, processors and/or other circuitry) including a horizontal small-dashed line indicating a corresponding use case budget threshold TH_{B}.

As previously discussed, it may be desired that the eye blinking in the user occurs substantially at the same time as the display or image artifact so that the user does not perceive the display or image artifact. The latency between the triggering of the eye blinking to the time the user blinks his/her eyes may be indicated as EB latency. The latency between the triggering of the victim throttling and the actual victim throttling when the display or image artifact occurs may be indicated as TT latency. If the EB latency is different than the TT latency, and both the eye blinking and victim throttling are triggered at the same time, then the eye blinking by the user may not occur at substantially the same time as the display or image artifact. In such case, the user would perceive the display or image artifact, and the user experience with the wearable device may be impacted.

In the example depicted in the graph of FIG. 4C, the EB latency is greater than the TT latency. Thus, in order for the eye blinking by the user to occur at substantially the same time as the display or image artifact, the triggering of the eye blinking should occur before (e.g., in anticipation of) the triggering of the victim throttling. To effectuate the triggering of the eye blinking before the triggering of the victim throttling, the main processor 315 uses the EB latency and the TT latency to stagger the eye blinking trigger and the victim throttling trigger.

For example, at time t₁, a request is provided to the main processor 315 for increased functionality/operation of one or more of the cores, peripherals, processors and/or other circuitry. In response to the request, the main processor 315 accesses the use case budget LUT 320, and determines that one or more victims is to be throttled. Then, at time t₂, the main processor 315 instructs the graphics core 335 (e.g., via the data bus 345) to generate and provide an image signal to the display 340 (e.g., via the data bus 345) that renders an image that induces eye blinking by the user. Then, at time t₃ (e.g., EB latency-TT latency after time t₂) the main processor 315 instructs one or more victims to throttle. Then, at time t₄, the user blinks his/her eyes at substantially the same time as the display or image artifact due to victim throttling. Thus, the user may not perceive the display or image artifact; and accordingly, the user experience with the wearable device 300 is improved.

In the previous current, temperature, use case load examples of FIGS. 4A-4C, respectively, the EB latency was greater than the TT latency. However, it shall be understood that the EB latency may be substantially equal to or less than the TT latency. In such case, the victim throttling may be triggered simultaneously with or before inducing eye blinking. Alternatively, if the EB latency is less than the TT latency, the victim throttling may be triggered substantially simultaneous with inducing eye blinking

FIG. 4D illustrates a block diagram of another example wearable device 400 in accordance with another aspect of the disclosure. As discussed in more detail herein, the wearable device 400 induces eye blinking substantially coincidental with victim throttling so that the eye blinking and the display or image artifacts occur at substantially the same time. If a user is blinking his or her eyes during the display of image artifacts, then the user will not observe the image artifacts; and thus, from a user's perspective, it is as if the image artifact did not occur. This would significantly improve the user experience because effectively the eye blinking masks the image artifact.

In particular, the wearable device 400 is similar to wearable device 300 previously discussed in detail, with similar elements identified by the same reference numbers with the exception that the most significant digit is a “4” in wearable device 400 compared to a “3” in wearable device 300. More specifically, the wearable device 400 includes a battery (Batt), a PMIC 405, a power/temperature monitor 410, a main processor 415, a use case budget LUT 420, one or more circuits, such as a set of cores (e.g., a multimedia core 425, a DSP core 430, and a graphic core 435), and a set of one or more peripherals, such as a display 440 and an eye tracker 450. The wearable device 400 further includes a data bus 445 communicatively coupling together the multimedia core 425, DSP core 430, graphics core 435, the display 440, the eye tracker 450, and the main processor 415. With the exception of the eye tracker, the operation of these elements with regard to inducing eye blinking and victim throttling have been previously discussed in detail with reference to wearable devices 200 and 300.

FIG. 4E illustrates a flow diagram of an example method 460 of inducing eye blinking in a time relationship with victim throttling in response to excess current or temperature in accordance with another aspect of the disclosure. The method 460 may be implemented by the main processor 415.

The method 460 includes the main processor 415 performing calibration of eye blinking latency (EB latency) based on a particular user and/or application (block 462). For example, this may entail the eye tracker 450 recognizing a new user based on characteristics of the user's eyes, and informing the main processor 415 of the new user via the data bus 445. The main processor 415 may then determine whether the application the user is running allows for inducing eye blinking associated with victim throttling. If not, the main processor 415 may not undergo the calibration operation of block 462. However, in this example, the application allows for eye blinking induction associated with victim throttling.

The EB calibration procedure may operate as follows: the main processor 415 may trigger eye blinking via the graphics core 435 and display 440 at a particular time t₁, and the eye tracker 450 detects the blinking of the eyes at a subsequent time t₂, and provides this information to the main processor 415. The main processor 415 may then determine the EB latency by taking the difference between times t₁ and t₂. Then main processor 415 may repeat the aforementioned eye blinking trigger and detection multiple times to obtain more data points on EB latency for the particular user so that a more accurate EB latency may be obtained. Although the calibration of the EB latency may be performed upon detecting a new user, it shall be understood that the EB latency calibration may be performed dynamically, periodically, or conditionally while a user uses the wearable device 400.

After EB latency calibration, the method 460 further includes the main processor 415 determining whether the sensed current or temperature has crossed the eye blinking threshold TH_{EB} (block 464). Then, according to the method 460, the main processor 415 determines whether the EB latency is greater than the TT latency (block 466). If the main processor 415 determines that the EB latency is greater than the TT latency in block 466, the method 460 includes the main processor 415 triggering the induction of eye blinking including sending an EB trigger instruction to the graphics core 435 via data bus 445 (block 468). Then, after a delay substantially equal or related to a difference Δ between the EB latency and the TT latency, the method 460 includes the main processor 415 triggering the throttling of one or more victims (block 470). If, in block 466, the main processor 415 determines that the EB latency is not greater than the TT latency, the method 460 includes the main processor 415 triggering eye blinking and victim throttling at substantially the same time (block 472).

FIG. 4F illustrates a flow diagram of an example method 480 of inducing eye blinking in a time relationship with victim throttling in response to anticipated excess use case load in accordance with another aspect of the disclosure. The method 480 may be implemented by the main processor 415. Although not shown in the example method 480, the main processor 415 may perform a calibration of eye blinking latency per user and application as discussed with reference to calibration operation specified block 462 of method 460.

The method 480 includes the main processor 415 determining a request to activate one or more cores would exceed the use case budget (block 482). Then, according to the method 480, the main processor 415 determines whether the EB latency is greater than the TT latency (block 484). If the main processor 415 determines that the EB latency is greater than the TT latency in block 484, the method 480 includes the main processor 415 triggering the induction of eye blinking including sending an EB trigger instruction to the graphics core 435 via data bus 445 (block 486). Then, after a delay substantially equal or related to a difference Δ between the EB latency and the TT latency, the method 480 includes the main processor 415 triggering the throttling of one or more victims (block 488). If, in block 484, the main processor 415 determines that the EB latency is not greater than the TT latency, the method 480 includes the main processor 415 triggering eye blinking and victim throttling at substantially the same time (block 490).

FIG. 5A illustrates timing diagrams related to scheduled eye blinking induction, victim throttling events, and actual eye blinking induction in accordance with another aspect of the disclosure. The wearable device 400 may induce eye blinking in a user based on a schedule (e.g., periodic eye blinking inductions) for eye health purposes. As previously discussed, the wearable device 400 may induce user eye blinking associated with victim throttling. The timing diagrams of FIG. 5A illustrates an example method of combining scheduled eye blinking induction with eye blinking induction due to victim throttling.

The x- or horizontal-axis of the timing diagrams represents time. The y- or vertical axis, from top to bottom, represents: the scheduled eye blinking induction (without considering eye blinking associated with victim throttling); example victim throttling events; and an actual eye blinking induction, which is based on the scheduled eye blinking induction and the victim throttling.

With reference to the top diagram, the scheduled eye blinking induction may include periodic eye blinking inductions with a period of T₀. For example, as shown, there is a first scheduled eye blinking induction at time t₁, a second scheduled eye blinking induction at time t₃ (where t₃=t₁+T₀), a third scheduled eye blinking induction at time t₅ (where t₅=t₁+2*T₀), a fourth scheduled eye blinking induction at time t₉ (where t₉=t₁+3*T₀), and so on. It shall be understood that the scheduled eye blinking induction may have a different scheduling, and need not be periodic, but could be in some other time-based pattern.

With reference to the middle diagram regarding victim throttling events, there is a first victim throttling at time t₂ due to a sensed current exceeding a maximum current rating threshold TH_{I}. There is a second victim throttling at time t₄ due to a use case load exceeding a use case budget threshold TH_{B}. There is a third victim throttling at time t₇ due to a sensed temperature exceeding a maximum temperature rating threshold TH_{T}.

With reference to the bottom diagram, the actual eye blinking induction may include an eye blinking induction at time t₁ (indicated with an “S” for scheduled), which coincides with the first scheduled eye blinking induction, as it is not associated with a victim throttling event. The actual eye blinking induction may further include a second eye blinking induction at approximately time t₂ (indicated with a “T” for throttling), which coincides with the first victim throttling at time t₂. Then the period T₀ is reset from the eye blinking induction due to victim throttling. Accordingly, the next scheduled eye blinking induction is at approximately time t₄ given by t₂+T₀.

As mentioned, the actual eye blinking induction includes an eye blinking induction at time t₄ (indicated with an “S/T”), as the eye blinking induction substantially coincides with a scheduled eye blinking induction and the second victim throttling at substantially time t₄. In other words, if a scheduled eye blinking induction substantially coincides with a victim throttling, there may not be a need for an additional or separate eye blinking induction. The actual eye blinking induction then includes the fourth eye blinking induction at approximately time t₆ (where t₆=t₄+T₀) (indicated with an “S”).

The actual eye blinking induction may further include a fifth eye blinking induction at approximately time t₈ (indicated with a “T”), which coincides with the third victim throttling at time t₇. Then again, the period T₀ is reset from the eye blinking induction due to the third victim throttling. Accordingly, the next scheduled eye blinking induction is at approximately time t₁₀ given by t₈+T₀.

FIG. 5B illustrates a flow diagram of an example method 500 of inducing eye blinking based on a scheduled eye blinking induction and victim throttling in accordance with another aspect of the disclosure. The method 500 may be implemented by the main processor 415 of wearable device 400.

In particular, the method 500 includes the main processor 415 determining that an eye blinking induction due to victim throttling is to be performed (block 505). The main processor 415 then determines whether the eye blinking induction coincides with a scheduled eye blinking induction (block 510). If the main processor 415 determines that the eye blinking induction associated with victim throttling coincides with a scheduled eye blinking induction in block 510, then the main processor 415 triggers the eye blinking induction in accordance with the current schedule (block 515). If, in block 510, the main processor 415 determines that the eye blinking induction associated with victim throttling does not coincides with a scheduled eye blinking induction, the main processor 415 triggers the eye blinking induction (block 520), and then modifies the current schedule of eye blinking induction (e.g., resets the period T₀) (block 525).

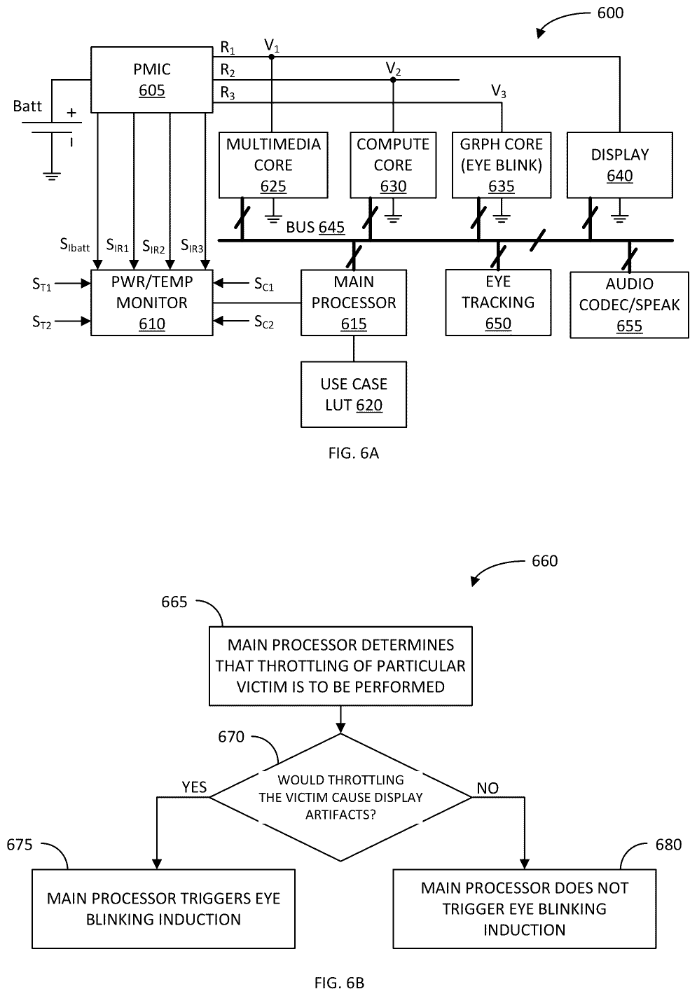

FIG. 6A illustrates a block diagram of another example wearable device 600 in accordance with another aspect of the disclosure. As previously discussed, the induction of eye blinking substantially coincidental with display or image artifacts due to victim throttling is done so that a user does not observe the artifacts; and thereby, the user experience with the wearable device is improved. However, if the particular victim that is throttled has no impact on the image being displayed (e.g., no display or image artifacts), then there is no need for inducing eye blinking for that particular victim. As discussed further herein, the wearable device 600 addresses the aforementioned.

In particular, the wearable device 600 is similar to wearable device 400 previously discussed in detail, with similar elements identified by the same reference numbers with the exception that the most significant digit is a “6” in wearable device 600 compared to a “4” in wearable device 400. More specifically, the wearable device 600 includes a battery (Batt), a PMIC 605, a power/temperature monitor 610, a main processor 615, a use case LUT 620, one or more circuits, such as a set of cores (e.g., a multimedia core 625, a DSP core 630, and a graphic core 635), and a set of one or more peripherals, such as a display 640 and an eye tracker 650.

Additionally, the wearable device 600 includes an audio codec/speaker 655. The wearable device 600 further includes a data bus 645 communicatively coupling together the multimedia core 625, DSP core 630, graphics core 635, the display 640, the eye tracker 650, the audio codec/speaker 655, and the main processor 615. With the exception of the audio codec/speaker 655, the operation of these elements with regard to inducing eye blinking and victim throttling have been previously discussed in detail with reference to wearable devices 200, 300, and 400. The audio codec/speaker 655 is an example of a victim that typically does not produce display or image artifacts when it is throttled.

FIG. 6B illustrates a flow diagram of an example method 660 of conditional eye blinking induction associated with victim throttling in accordance with another aspect of the disclosure. The method 660 may be implemented by main processor 615.

The method 660 includes the main processor 615 determining that throttling of a particular victim is to be performed (block 665). The method 660 further includes the main processor 615 determining whether throttling the particular victim causes display or image artifacts (block 670). The main processor 615 may obtain such information from a look-up table (LUT) indicating which victims cause display or image artifacts, and implicitly which victims does not, or vice-versa. If, in block 670, the main processor 615 determines that the particular victim causes display or image artifacts, the main processor 615 triggers eye blinking in accordance with the methods previously discussed (block 675). If, in block 670, the main processor 615 determines that the particular victim does not cause display or image artifacts, the main processor 615 does not trigger eye blinking associated with the throttling of such victim (block 680).

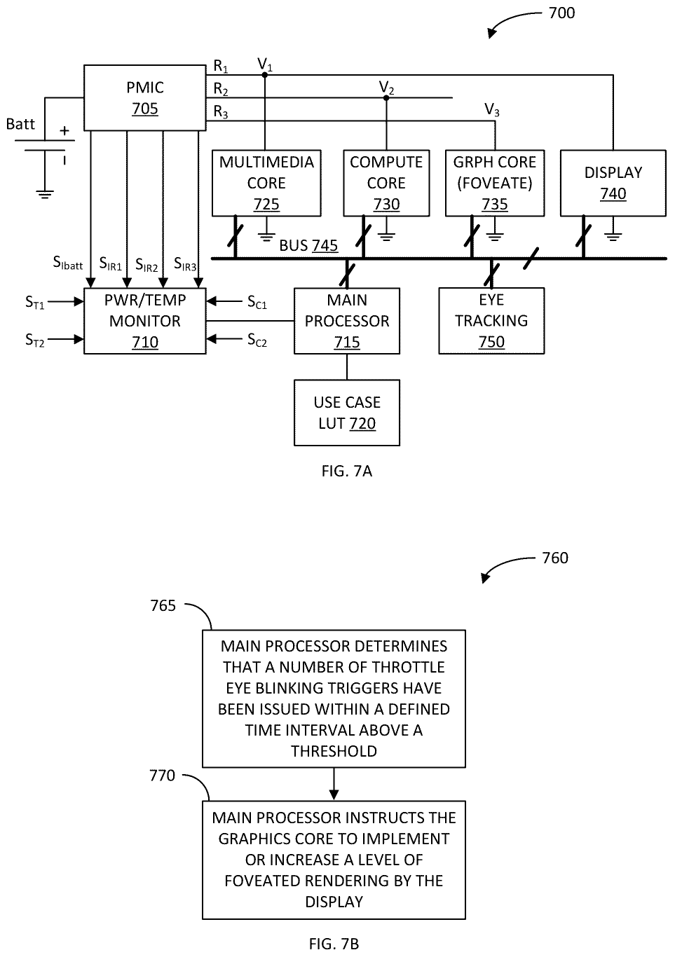

FIG. 7A illustrates a block diagram of another example wearable device 700 in accordance with another aspect of the disclosure. In some cases, where there may be relatively high fluctuations in currents (e.g., battery current, rail current, core current, etc.) due to one or more cores, peripherals, processors or other circuitry performing operations that produce such high current fluctuations, eye blinking may be induced very frequently, which may have the adverse effect of impacting user experience. In such case, a main processor may take actions to reduce the average current so as to reduce frequent eye blinking triggers. As discussed further herein, the wearable device 700 addresses the aforementioned.

In particular, the wearable device 700 is similar to wearable devices 400 previously discussed in detail, with similar elements identified by the same reference numbers with the exception that the most significant digit is a “7” in wearable device 700 compared to a “4” in wearable device 400. More specifically, the wearable device 700 includes a battery (Batt), a PMIC 705, a power/temperature monitor 710, a main processor 715, a use case budget LUT 720, one or more circuits, such as a set of cores (e.g., a multimedia core 725, a DSP core 730, and a graphic core 735), and a set of one or more peripherals, such as a display 740 and an eye tracker 750. As previously discussed, a data bus 745 may communicatively couple together the multimedia core 725, DSP core 730, graphics core 735, the display 740, the eye tracker 750, and the main processor 715.

In wearable device 700, the graphics core 735 is capable of foveated rendering of images by the display 740. A foveated image is one where the resolution of a portion or region of an image at which the user is directly looking is higher than the resolution of one or more other portions or regions (e.g., the peripheral portion of the image) at which the user is not directly looking. If frequent eye blinking triggers are detected by the main processor 715, the main processor 715 may cause the graphics core 735 (e.g., via the data bus 745) to implement foveated rendering of images by the display 740. This causes a reduction in the current due to the lower resolution at the non-foveated portion of the image, which reduces the average current; and thereby, reduces the frequent eye blinking triggers due to victim throttling.

FIG. 7B illustrates a flow diagram of an example method 760 of initiating or increasing foveate rendering in response to frequent eye blinking inductions in response to victim throttling in accordance with another aspect of the disclosure. The method 760 may be implemented by the main processor 715.

The method 760 includes the main processor 715 determining that a number of throttle-based eye blinking triggers have been issued within a defined time interval exceeding a certain threshold (block 765). The threshold may indicate the frequency of eye blinking triggers that would significantly impact the user experience. The method 760 further includes the main processor 715 instructing the graphics core 735 via the data bus 745 to implement or increase a level of foveated rendering by the display 740 (block 770). Increasing the level of foveated rendering by the display 740 may include increasing the non-foveated portion relative to the foveated portion of the image, and/or further reducing the resolution of the non-foveated portion of the image. As discussed, this reduces the frequent eye blinking triggers due to victim throttling, and improves the user experience for the user.

Some of the components described herein, such as one or more of the subsystems, thermal controllers, and communication interfaces, may be implemented using a processor. A processor, as used herein, may be any dedicated circuit, processor-based hardware, a processing core of a system on chip (SOC), etc. Hardware examples of a processor may include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described throughout this disclosure.

The processor may be coupled to memory (e.g., generally a computer-readable media or medium), such as a magnetic storage device (e.g., hard disk, floppy disk, magnetic strip), an optical disk (e.g., a compact disc (CD) or a digital versatile disc (DVD)), a smart card, a flash memory device (e.g., a card, a stick, or a key drive), a random access memory (RAM), a read only memory (ROM), a programmable ROM (PROM), an erasable PROM (EPROM), an electrically erasable PROM (EEPROM), a register, a removable disk, and any other suitable medium for storing software and/or instructions that may be accessed and read by a computer. The memory may store computer-executable code (e.g., software). Software shall be construed broadly to mean instructions, instruction sets, code, code segments, program code, programs, subprograms, software modules, applications, software applications, software packages, routines, subroutines, objects, executables, threads of execution, procedures/processes, functions, etc., whether referred to as software, firmware, middleware, microcode, hardware description language, or otherwise.

The following provides an overview of aspects of the present disclosure:

Aspect 1: A wearable device, comprising: a display; one or more circuits coupled to the display; and a processor configured to: trigger a throttling of the one or more circuits or the display; and trigger the display to generate a first image that induces eye blinking in a user associated with the throttling of the one or more circuits or the display.

Aspect 2: The wearable device of aspect 1, wherein the processor is configured to trigger the throttling of the one or more circuits or the display in response to a current exceeding a current threshold.

Aspect 3: The wearable device of aspect 2, wherein the wearable device comprises a battery coupled to the one or more circuits and the display, and wherein the current comprises a battery current supplied by the battery.

Aspect 4: The wearable device of aspect 2 or 3, wherein the wearable device comprises a power management integrated circuit (PMIC) coupled to one or more power rails, wherein the one or more power rails are coupled to the one or more circuits and the display, and wherein the current comprises one or more rail currents supplied by the one or more power rails, respectively.

Aspect 5: The wearable device of any one of aspects 2-4, wherein the current comprises a current drawn by the one or more circuits.

Aspect 6: The wearable device of any one of aspects 2-5, wherein the processor is configured to trigger the throttling of the one or more circuits or the display in response to a temperature exceeding a temperature threshold.

Aspect 7: The wearable device of aspect 6, further comprising one or more temperature sensors configured to sense one or more skin temperatures of the user, wherein the temperature comprises the one or more skin temperatures.

Aspect 8: The wearable device of aspect 6 or 7, further comprising one or more temperature sensors configured to sense one or more circuit temperatures proximate the one or more circuits, wherein the temperature comprises the one or more circuit temperatures.

Aspect 9: The wearable device of any one of aspects 1-8, wherein the processor is configured to trigger the throttling of the one or more circuits or the display in response to a request for increased use case load.

Aspect 10: The wearable device of any one of aspects 1-9, wherein the first image that induces eye blinking in the user comprises an image including at least a region that is blurred.

Aspect 11: The wearable device of any one of aspects 1-10, wherein the processor is configured to trigger the display to generate the first image that induces eye blinking in the user such that the user blinks his/her eyes substantially coincidental with the display producing a second image that includes an artifact due to the throttling of the one or more circuits or the display.

Aspect 12: The wearable device of aspect 11, wherein the artifact comprises a display or image distortion, a glitch, a jitter, or a display or image tearing.

Aspect 13: The wearable device of any one of aspects 1-12, wherein the processor is configured to trigger the display to generate the first image that induces eye blinking in the user in a time relationship with the triggering of the throttling of the one or more circuits or the display.

Aspect 14: The wearable device of any one of aspects 1-13, wherein: a first latency exists between the triggering of the display to generate the first image and a calibrated or estimated time when the user blinks his/her eyes; and a second latency exists between the triggering of the throttling of the one or more circuits or the display and the display producing a second image with an artifact as a result of the throttling of the one or more circuits or the display.

Aspect 15: The wearable device of aspect 14, wherein the processor is configured to trigger the display to generate the first image and trigger the throttling of the one or more circuits or the display based on a difference between the first and second latencies.

Aspect 16: The wearable device of aspect 15, wherein the processor is configured to trigger the display to generate the first image and the throttling of the one or more circuits or the display at substantially the same time if the difference between the first and second latencies is substantially zero (0) or negative.

Aspect 17: The wearable device of aspect 15, wherein the processor is configured to trigger the display to generate the first image at a time interval before the throttling of the one or more circuits or the display related to the difference between the first and second latencies.

Aspect 18: The wearable device of aspect 17, wherein: the processor is configured to trigger the display to generate the first image that induces eye blinking in the user in response to a current crossing a first current threshold; the processor is configured to trigger the throttling of the one or more circuits or the display in response to the current crossing a second current threshold; wherein a difference between the first and second current thresholds is related to the difference between the first and second latencies.

Aspect 19: The wearable device of aspect 17 or 18, wherein: the processor is configured to trigger the display to generate the first image that induces eye blinking in the user in response to a temperature crossing a first temperature threshold; the processor is configured to trigger the throttling of the one or more circuits or the display in response to the temperature crossing a second temperature threshold; wherein a difference between the first and second temperature thresholds is related to the difference between the first and second latencies.

Aspect 20: The wearable device of any one of aspects 1-19, wherein: the display is configured to generate a scheduled set of images to induce eye blinking in the user in response to the scheduled set of images; and wherein the processor is configured to trigger the display to generate the first image that induces eye blinking in the user in addition to the set of images that induces eye blinking in the user.

Aspect 21: The wearable device of aspect 20, wherein the processor is configured to modify the scheduled set of images in response to triggering the display to generate the first image.

Aspect 22: The wearable device of aspect 21, wherein the scheduled set of images are generated based on a periodic, and wherein the processor is configured to reset the period of the scheduled set of images based on the first image.

Aspect 23: The wearable device of any one of aspects 1-22, wherein: the display is configured to generate a scheduled set of images to induce eye blinking in the user in response to the scheduled set of images; and wherein the processor is configured to treat the first image as part of the scheduled set of images in response to the first image substantially coinciding with one of the scheduled set of images.

Aspect 24: The wearable device of any one of aspects 1-23, wherein the processor is configured to: determine whether the throttling of the one or more circuits is deemed to cause the display to generate a second image with an artifact; and not trigger the display to generate the first image in response to determining that the one or more circuits is not deemed to cause the display to generate the second image with the artifact.

Aspect 25: The wearable device of aspect 24, wherein the one or more circuits that is deemed not to cause the display to generate the second image with the artifact includes an audio circuit.

Aspect 26: The wearable device of aspect 1, wherein the processor is configured to: trigger the display to generate a set of images that induces eye blinking in the user based on a set of throttling events of the one or more circuits or the display, respectively; determine a number of the set of images generated within a defined time interval; and trigger the display to generate a second set of images with foveated rendering or increased foveated rendering in response to the number of the set of images generated within the defined time interval exceeding a threshold.

Aspect 27: A method of operating a wearable device, comprising: triggering a throttling of one or more circuits; and inducing eye blinking in a user of the wearable device such that the eye blinking occurs substantially coincidental with a display generating an image including an artifact as a result of the throttling of one or more circuits.

Aspect 28: The method of aspect 27, wherein inducing the eye blinking in the user occurs before the triggering of the throttling of the one or more circuits.

Aspect 29: An apparatus, comprising: means for triggering a throttling of one or more circuits; and means for inducing eye blinking in a user such that the eye blinking occurs substantially coincidental with a display generating an image including an artifact as a result of the throttling of one or more circuits.

Aspect 30: An augmented or virtual reality eyewear, comprising: a display; a graphics core coupled to the display; a multimedia core coupled to the graphics core; a digital signal processor (DSP) core coupled to the multimedia core; and a processor configured to: trigger a throttling of the one or more of the multimedia core, the graphics core, the DSP core, or the display; and trigger the display to generate a first image that induces eye blinking in a user based on the throttling of the one or more of the multimedia core, the graphics core, the DSP core, or the display.

The previous description of the disclosure is provided to enable any person skilled in the art to make or use the disclosure. Various modifications to the disclosure will be readily apparent to those skilled in the art, and the generic principles defined herein may be applied to other variations without departing from the spirit or scope of the disclosure. Thus, the disclosure is not intended to be limited to the examples described herein but is to be accorded the widest scope consistent with the principles and novel features disclosed herein.

What is claimed:

1. A wearable device, comprising:
a display;
one or more circuits coupled to the display; and
a processor configured to:
trigger a throttling of the one or more circuits or the display; and
trigger the display to generate a first image that induces eye blinking in a user associated with the throttling of the one or more circuits or the display.

2. The wearable device of claim 1, wherein the processor is configured to trigger the throttling of the one or more circuits or the display in response to a current exceeding a current threshold.

3. The wearable device of claim 2, wherein the wearable device comprises a battery coupled to the one or more circuits and the display, and wherein the current comprises a battery current supplied by the battery.

4. The wearable device of claim 2, wherein the wearable device comprises a power management integrated circuit (PMIC) coupled to one or more power rails, wherein the one or more power rails are coupled to the one or more circuits and the display, and wherein the current comprises one or more rail currents supplied by the one or more power rails, respectively.

5. The wearable device of claim 2, wherein the current comprises a current drawn by the one or more circuits.

6. The wearable device of claim 1, wherein the processor is configured to trigger the throttling of the one or more circuits or the display in response to a temperature exceeding a temperature threshold.

7. The wearable device of claim 6, further comprising one or more temperature sensors configured to sense one or more skin temperatures of the user, wherein the temperature comprises the one or more skin temperatures.

8. The wearable device of claim 6, further comprising one or more temperature sensors configured to sense one or more circuit temperatures proximate the one or more circuits, wherein the temperature comprises the one or more circuit temperatures.

9. The wearable device of claim 1, wherein the processor is configured to trigger the throttling of the one or more circuits or the display in response to a request for increased use case load.

10. The wearable device of claim 1, wherein the first image that induces eye blinking in the user comprises an image including at least a region that is blurred.

11. The wearable device of claim 1, wherein the processor is configured to trigger the display to generate the first image that induces eye blinking in the user such that the user blinks his/her eyes substantially coincidental with the display producing a second image that includes an artifact due to the throttling of the one or more circuits or the display.

12. The wearable device of claim 11, wherein the artifact comprises a display or image distortion, a glitch, a jitter, or a display or image tearing.

13. The wearable device of claim 1, wherein the processor is configured to trigger the display to generate the first image that induces eye blinking in the user in a time relationship with the triggering of the throttling of the one or more circuits or the display.

14. The wearable device of claim 1, wherein:
a first latency exists between the triggering of the display to generate the first image and a calibrated or estimated time when the user blinks his/her eyes; and
a second latency exists between the triggering of the throttling of the one or more circuits or the display and the display producing a second image with an artifact as a result of the throttling of the one or more circuits or the display.

15. The wearable device of claim 14, wherein the processor is configured to trigger the display to generate the first image and trigger the throttling of the one or more circuits or the display based on a difference between the first and second latencies.

16. The wearable device of claim 15, wherein the processor is configured to trigger the display to generate the first image and trigger the throttling of the one or more circuits or the display at substantially the same time if the difference between the first and second latencies is substantially zero (0) or negative.

17. The wearable device of claim 15, wherein the processor is configured to trigger the display to generate the first image at a time interval before the throttling of the one or more circuits or the display related to the difference between the first and second latencies.

18. The wearable device of claim 17, wherein:
the processor is configured to trigger the display to generate the first image that induces eye blinking in the user in response to a current crossing a first current threshold;
the processor is configured to trigger the throttling of the one or more circuits or the display in response to the current crossing a second current threshold;
wherein a difference between the first and second current thresholds is related to the difference between the first and second latencies.

19. The wearable device of claim 17, wherein:
the processor is configured to trigger the display to generate the first image that induces eye blinking in the user in response to a temperature crossing a first temperature threshold;
the processor is configured to trigger the throttling of the one or more circuits or the display in response to the temperature crossing a second temperature threshold;
wherein a difference between the first and second temperature thresholds is related to the difference between the first and second latencies.

20. The wearable device of claim 1, wherein:
the display is configured to generate a scheduled set of images to induce eye blinking in the user in response to the scheduled set of images; and
wherein the processor is configured to trigger the display to generate the first image that induces eye blinking in the user in addition to the set of images that induces eye blinking in the user.

21. The wearable device of claim 20, wherein the processor is configured to modify the scheduled set of images in response to triggering the display to generate the first image.

22. The wearable device of claim 21, wherein the scheduled set of images are generated based on a periodic, and wherein the processor is configured to reset the period of the scheduled set of images based on the first image.

23. The wearable device of claim 1, wherein:
the display is configured to generate a scheduled set of images to induce eye blinking in the user in response to the scheduled set of images; and
wherein the processor is configured to treat the first image as part of the scheduled set of images in response to the first image substantially coinciding with one of the scheduled set of images.

24. The wearable device of claim 1, wherein the processor is configured to:
determine whether the throttling of the one or more circuits is deemed to cause the display to generate a second image with an artifact; and
not trigger the display to generate the first image in response to determining that the one or more circuits is not deemed to cause the display to generate the second image with the artifact.

25. The wearable device of claim 24, wherein the one or more circuits that is not deemed to cause the display to generate the second image with the artifact includes an audio circuit.

26. The wearable device of claim 1, wherein the processor is configured to:
trigger the display to generate a set of images that induces eye blinking in the user based on a set of throttling events of the one or more circuits or the display, respectively;
determine a number of the set of images generated within a defined time interval; and
trigger the display to generate a second set of images with foveated rendering or increased foveated rendering in response to the number of the set of images generated within the defined time interval exceeding a threshold.

27. A method of operating a wearable device, comprising:
triggering a throttling of one or more circuits; and
inducing eye blinking in a user of the wearable device such that the eye blinking occurs substantially coincidental with a display generating an image including an artifact as a result of the throttling of one or more circuits.

28. The method of claim 27, wherein inducing the eye blinking in the user occurs before the triggering of the throttling of the one or more circuits.

29. An apparatus, comprising:
means for triggering a throttling of one or more circuits; and
means for inducing eye blinking in a user such that the eye blinking occurs substantially coincidental with a display generating an image including an artifact as a result of the throttling of one or more circuits.

30. An augmented or virtual reality eyewear, comprising:
a display;
a graphics core coupled to the display;
a multimedia core coupled to the graphics core;
a digital signal processor (DSP) core coupled to the multimedia core; and
a processor configured to:
trigger a throttling of the one or more of the multimedia core, the graphics core, the DSP core, or the display; and
trigger the display to generate a first image that induces eye blinking in a user associated with the throttling of the one or more of the multimedia core, the graphics core, the DSP core, or the display.
